(19) United States
(12) Patent Application Publication
(10) Pub. No.: US 2017/0252104 A1
(43) Pub. Date: Sep. 7, 2017

(54) ABLATION DEVICE

(71) Applicant: OLYMPUS CORPORATION, Tokyo (JP)

(72) Inventor: Naoya SUGIMOTO, Tokyo (JP)

(73) Assignee: OLYMPUS CORPORATION, Tokyo (JP)

(21) Appl. No.: 15/598,917

(22) Filed: May 18, 2017

Related U.S. Application Data
(63) Continuation of application No. PCT/JP2014/081499, filed on Nov. 28, 2014.

(51) Int. Cl. A61B 18/14; A61B 18/12

(52) U.S. Cl. CPC A61B 18/1492; A61B 18/1206; A61B 2018/00577

(57) ABSTRACT

Provided is an ablation device including a narrow insertion section insertable into a body, a distal end section provided at a distal end of the insertion section and having a narrow cauterizing surface that is formed in a longitudinal direction in one area in a circumferential direction and that releases energy to biological tissue, and a radiopaque marking section provided at the distal end section and substantially parallel to the cauterizing surface. The marking section has a three-dimensional shape such that projection shapes obtained when projected from different sides in a radial direction of the distal end section are different from each other.

### CROSS-REFERENCE TO RELATED APPLICATIONS

This is a continuation of International Application PCT/JP2014/081499, with an international filing date of Nov. 28, 2014, which is hereby incorporated by reference herein in its entirety. This application claims the benefit of International Application PCT/JP2014/081499.

### TECHNICAL FIELD

The present invention relates to ablation devices.

### BACKGROUND ART

In treatment of arrhythmia in the related art, ablation devices that linearly cauterize the cardiac surfaces are used (for example, see Patent Literature 1 and Non Patent Literature 1). In atrial cells, an abnormal electric signal is generated mainly near the pulmonary vein of the left atrium. Therefore, by linearly cauterizing the left atrium so as to surround the base of the pulmonary vein, the abnormal electric signal can be prevented from being transmitted from the pulmonary vein to the surrounding area thereof.

A known minimally-invasive heart surgery method involves percutaneously inserting a treatment device into the pericardial cavity through the pericardium from the xiphoid process.

### CITATION LIST

### Patent Literature

{PTL 1}
Japanese Translation of PCT International Application, Publication No. 2003-527188

### Non Patent Literature

{NPL 1}
“COBRA Adhere XL”, [online], Estech Corp., [Search Date: Sep. 26, 2014], Internet <http://www.estech.com/node/sites/default/files/datasheets/460-11684-LIT_Rev%20E%20COBRA%20Adhere%20XL%20Data%20Sheet-web.pdf>

### SUMMARY OF INVENTION

### Technical Problem

Electrodes for supplying high-frequency current to the heart are provided not around the entire circumference of the ablation device but only in one area in the circumferential direction. Therefore, in order to reliably cauterize the cardiac surface, the orientation of the ablation device around the longitudinal axis has to be adjusted so that the electrodes come into contact with the cardiac surface.

An object of the present invention is to provide an ablation device that allows the orientation of a cauterizing surface percutaneously inserted in the body to be externally recognized so that tissue can be reliably cauterized.

### Solution to Problem

A first aspect of the present invention provides an ablation device including a narrow insertion section insertable into a body, a distal end section provided at a distal end of the insertion section and having a narrow cauterizing surface that is formed in a longitudinal direction in one area in a circumferential direction and that releases energy to biological tissue, and a radiopaque marking section provided at the distal end section and substantially parallel to the cauterizing surface. The marking section has a three-dimensional shape such that projection shapes obtained when projected from different sides in a radial direction of the distal end section are different from each other.

In the above aspect, the marking section may be provided at the cauterizing surface or may be provided at a side surface of the distal end section that is substantially orthogonal to the cauterizing surface.

In the above aspect, the marking section may include a plurality of markers arranged in the longitudinal direction in a row at an intervals.

In the above aspect, the ablation device may further include an insulation member having electrical insulation properties and accommodating the distal end section and the insertion section in a movable manner in the longitudinal direction.

### BRIEF DESCRIPTION OF DRAWINGS

FIG. 1 illustrates the overall configuration of an ablation device according to an embodiment of the present invention.

FIG. 2A illustrates a side view of a distal end section of the ablation device in FIG. 1, as viewed from a cauterizing surface side.

FIG. 2B illustrates a cross-sectional view of a distal end section of the ablation device in FIG. 1 taken along line II-II.

FIG. 3 illustrates a method of using the ablation device in FIG. 1.

FIG. 4 illustrates a modification of the type of a marking section and is a side view of the distal end section, as viewed from the cauterizing surface side.

FIG. 5 illustrates another modification of the type of the marking section and is a side view of the distal end section, as viewed from the cauterizing surface side.

FIG. 6 illustrates another modification of the type of the marking section and is a side view of the distal end section, as viewed from the cauterizing surface side.

FIG. 7 illustrates another modification of the type of the marking section and is a side view of the distal end section, as viewed from the cauterizing surface side.

FIG. 8 illustrates a modification of the disposition of the marking section and is a cross-sectional view of the distal end section.

FIG. 9 illustrates a method of using the ablation device in FIG. 8.

FIG. 10 illustrates another modification of the disposition of the marking section and is a cross-sectional view of the distal end section.

FIG. 11 illustrates another modification of the type and the disposition of the marking section and is a cross-sectional view of the distal end section.

FIG. 12 is a side view illustrating an insulation member provided in the ablation device in FIG. 1.

### DESCRIPTION OF EMBODIMENTS

An ablation device 1 according to an embodiment of the present invention will be described below with reference to the drawings.

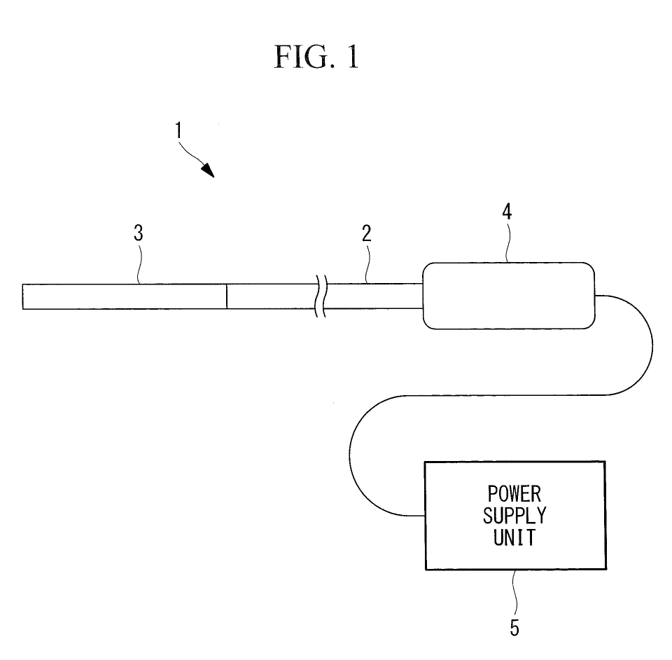

As shown in FIG. 1, the ablation device 1 according to this embodiment includes a narrow insertion section 2, a distal end section 3 that is connected to the distal end of the insertion section 2 and that is used for cauterizing tissue, a handle 4 connected to the base end of the insertion section 2, and a power supply unit 5 that supplies high-frequency current to the distal end section 3 for cauterization.

The insertion section 2 and the distal end section 3 have flexibility such that they are bendable in conformity to the shape of surrounding tissue, and are percutaneously insertable into the pericardial cavity.

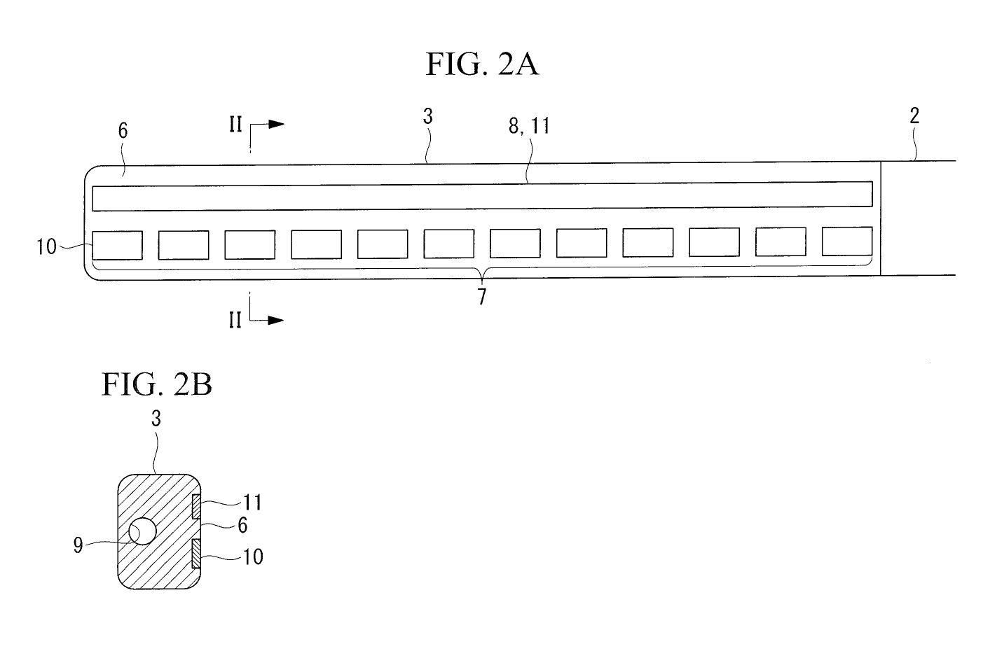

FIGS. 2A and 2B illustrate the structure of the distal end section 3. As shown in FIGS. 2A and 2B, the distal end section 3 has a flat cauterizing surface 6 formed in one area thereof in the circumferential direction. The cauterizing surface 6 is provided with a cauterizing section (marking section) 7 and a marking section 8. Reference sign 9 denotes a guide wire hole that extends through the distal end section 3 and the insertion section 2 in the longitudinal direction from the distal end surface of the distal end section 3 to the base end of the insertion section 2.

The cauterizing surface 6 has a narrow rectangular shape extending in the longitudinal direction.

The cauterizing section 7 is constituted of a plurality of electrodes 10 spaced apart from one another and arranged in a row in the longitudinal direction. In the drawings, only one of the plurality of electrodes is given the reference sign 10. The electrodes 10, which are composed of a conductive material, such as metal, have X-ray impermeability. The electrodes 10 are exposed to the outside so as to be directly contactable with tissue. Moreover, the electrodes 10 are electrically connected to the power supply unit 5 by wires that extend inside the distal end section 3, the insertion section 2, and the handle 4 to the power supply unit 5.

The marking section 8 is constituted of a single flat and narrow strip-shaped marker 11 composed of a radiopaque material, such as platinum or palladium. The marker 11 is provided parallel to the cauterizing surface 6. Furthermore, the marker 11 is spaced apart from the cauterizing section 7 and is provided parallel to the cauterizing section 7 along the entire length of the cauterizing section 7.

Next, the operation of the ablation device 1 having the above-described configuration will be described.

The ablation device 1 according to this embodiment is used in treatment that involves directly ablating the cardiac surface from the outside.

First, for example, a guide wire is inserted into the body from below the xiphoid process and is pierced through the pericardium so as to be inserted into the pericardial cavity. Then, the guide wire is inserted into the guide wire hole 9, and the distal end section 3 and the insertion section 2 are moved forward along the guide wire, thereby introducing the distal end section 3 and the insertion section 2 into the pericardial cavity. The guide wire, the distal end section 3, and the insertion section 2 are operated inside the body while observing the patient's thorax by using an X-ray fluoroscope.

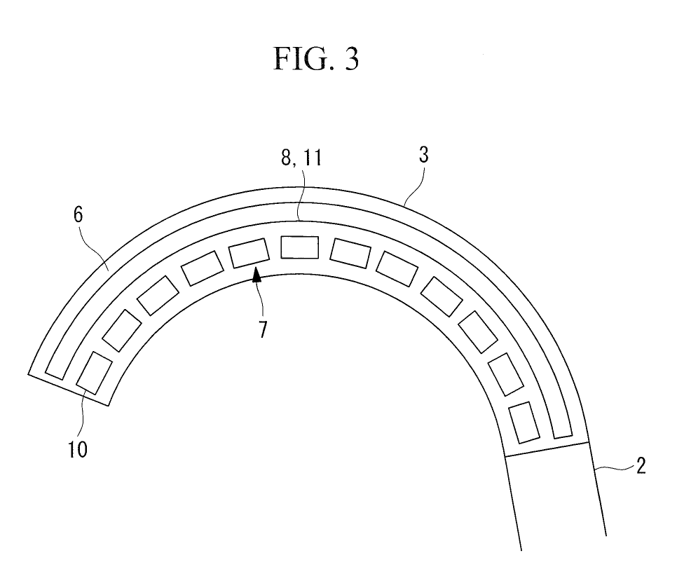

Then, for example, as shown in FIG. 3, the distal end section 3 is bent so as to surround a site that is causing arrhythmia of the heart, and a high-frequency current is supplied from the power supply unit 5 to the electrodes 10. In FIG. 3, the heart is located at the near side of the drawing. Thus, cardiac tissue is linearly cauterized between the site and the surrounding area thereof, so that transmission of an abnormal signal from the site to the surrounding area can be blocked.

The relationship between the orientation (i.e., the rotational angle around the longitudinal axis) of the distal end section 3 inside the pericardial cavity and the shapes of projection images of the cauterizing section 7 and the marking section 8 in an X-ray fluoroscopic image will now be described.

In a case where the abdomen of a patient lying facing upward is to be observed from top to bottom in the vertical direction by using an X-ray fluoroscope, when the cauterizing surface 6 is facing vertically upward, the shapes of the projection images of the cauterizing section 7 and the marking section 8 in the X-ray fluoroscopic image are identical to the shapes of the cauterizing section 7 and the marking section 8 shown in FIG. 2A.

When the cauterizing surface 6 is facing vertically downward, the shapes of the projection images of the cauterizing section 7 and the marking section 8 in the X-ray fluoroscopic image are such that the cauterizing section 7 and the marking section 8 in FIG. 2A are inverted in the width direction (i.e., the positions of the cauterizing section 7 and the marking section 8 are interchanged).

When the cauterizing surface 6 is tilted relative to the vertical direction, the shapes of the projection images of the cauterizing section 7 and the marking section 8 in the X-ray fluoroscopic image are such that the cauterizing section 7 and the marking section 8 in FIG. 2A are compressed in the width direction.

Accordingly, in this embodiment, the cauterizing section 7 and the marking section 8 collectively have a three-dimensional shape such that, when they are projected in the radial direction of the distal end section 3 from the cauterizing surface 6 side, the projection shapes thereof are asymmetrical in the width direction, which intersects the longitudinal axis of the distal end section 3. Therefore, the shapes of the projection images of the combination of the cauterizing section 7 and the marking section 8 in the X-ray fluoroscopic image vary depending on from which side, in the radial direction of the distal end section 3, observation is performed using the X-ray fluoroscope.

Accordingly, a surgeon can recognize the orientation of the distal end section 3 inside the pericardial cavity from the shapes of the projection images of the combination of the cauterizing section 7 and the marking section 8 in the X-ray fluoroscopic image. This is advantageous in that the surgeon can effectively treat a disease, such as arrhythmia, by properly adjusting the orientation of the distal end section 3 so that the cauterizing surface 6 comes into contact with the cardiac surface, and by reliably supplying high-frequency current from the electrodes 10 to the cardiac tissue.

The distal end section 3 is provided with the strip-shaped marking section 8 extending continuously in the longitudinal direction. This marking section 8 deforms spirally as the distal end section 3 is twisted around the longitudinal axis. This advantageous in that the surgeon can recognize whether or not the distal end section 3 is twisted and can recognize the degree of twisting from the overall shape of the marking section 8 in the X-ray fluoroscopic image. Moreover, the cauterizing surface 6 provided with the cauterizing section 7 is also provided with the marking section 8. This is advantageous in that the orientation of the cauterizing surface 6 relative to the cardiac tissue can be recognized more accurately.

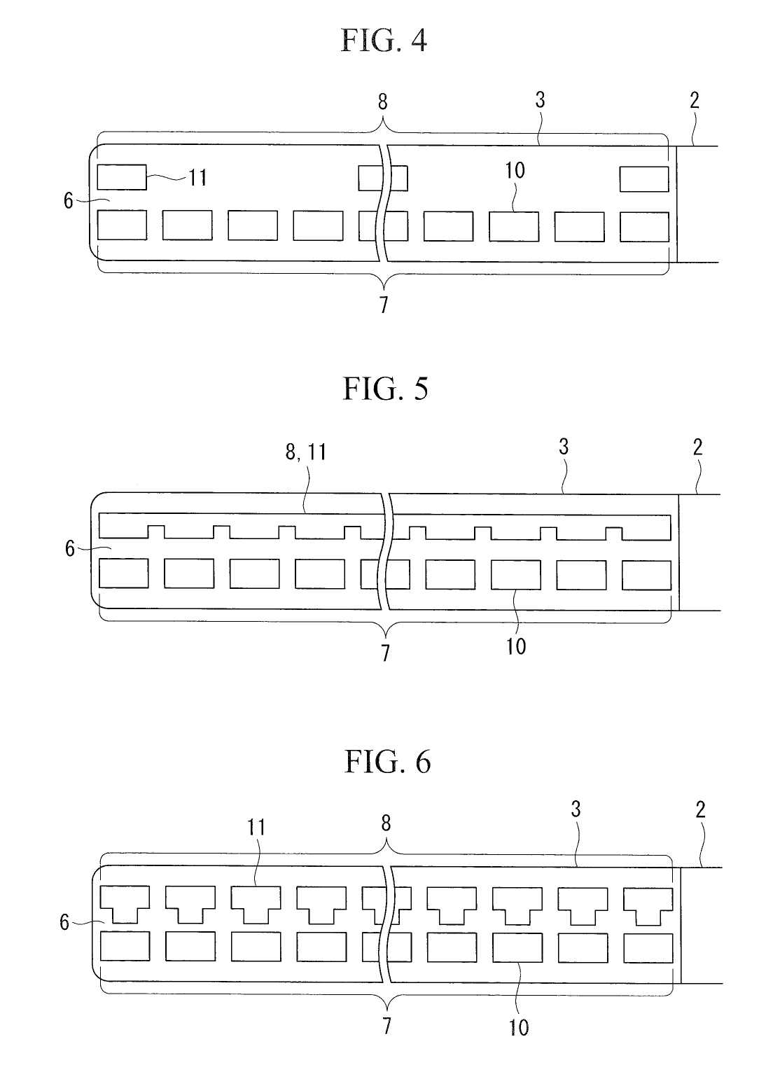

In this embodiment, the marking section 8 is constituted of the single marker 11. Alternatively, as shown in FIG. 4, the marking section 8 may be constituted of a plurality of markers 11 spaced apart from one another in the longitudinal direction and arranged in a row. Accordingly, the plurality of markers 11 can also function as a scale indicating positions in the longitudinal direction of the distal end section 3 so that, for example, it can be quantitatively recognized which position in the distal end section 3 is satisfactorily in contact with the heart. In this modification, it is preferable that the distance between the markers 11 in the longitudinal direction be different from the distance between the electrodes 10 in the longitudinal direction so that the projection image of the marking section 8 and the projection image of the cauterizing section 7 can be easily distinguished from each other in the X-ray fluoroscopic image.

In this embodiment, the cauterizing section 7 and the marking section 8 are both composed of a radiopaque material, and the orientation of the distal end section 3 is identified based on the projection images of the combination of the cauterizing section 7 and the marking section 8. Alternatively, as shown in FIGS. 5 to 7, the orientation of the distal end section 3 may be identifiable based on the projection image of the marking section 8 alone or the projection image of the cauterizing section 7 alone.

In a modification shown in FIG. 5, the marking section 8 is similar to the marking section 8 in FIGS. 2A and 2B in that it is constituted of a single flat and narrow strip-shaped marker 11, but the marker 11 has an asymmetrical shape in the width direction. Specifically, of two side surfaces of the marker 11 opposite each other in the width direction, one is flat, whereas the other has an irregular shape. The projection shape of such a marking section 8 when the cauterizing surface 6 is viewed from the front is inverted when the cauterizing surface 6 is viewed from the back. Therefore, the orientation of the distal end section 3 can be recognized based on the projection image of the marking section 8 alone in the X-ray fluoroscopic image.

FIG. 6 illustrates a modification of the marking section 8 in FIG. 5. The marking section 8 in FIG. 6 is constituted of a plurality of markers 11. Of two side surfaces of each marker 11 opposite each other in the width direction, one is flat, whereas the other has an irregular shape. The projection shape of such a marking section 8 when the cauterizing surface 6 is viewed from the front is inverted when the cauterizing surface 6 is viewed from the back. Therefore, the orientation of the distal end section 3 can be recognized based on the projection image of the marking section 8 alone in the X-ray fluoroscopic image. In this modification, the cauterizing section 7 does not have to be radiopacity.

In the case of the marking section 8 in FIGS. 2A and 2B, it is necessary to observe the entire marking section 8 to determine whether the distal end section 3 is twisted. In contrast, in the case of the marking section 8 in this modification, the twisting of the distal end section 3 can be readily recognized based on the shape of the projection image of the irregular section even from one area in the longitudinal direction.

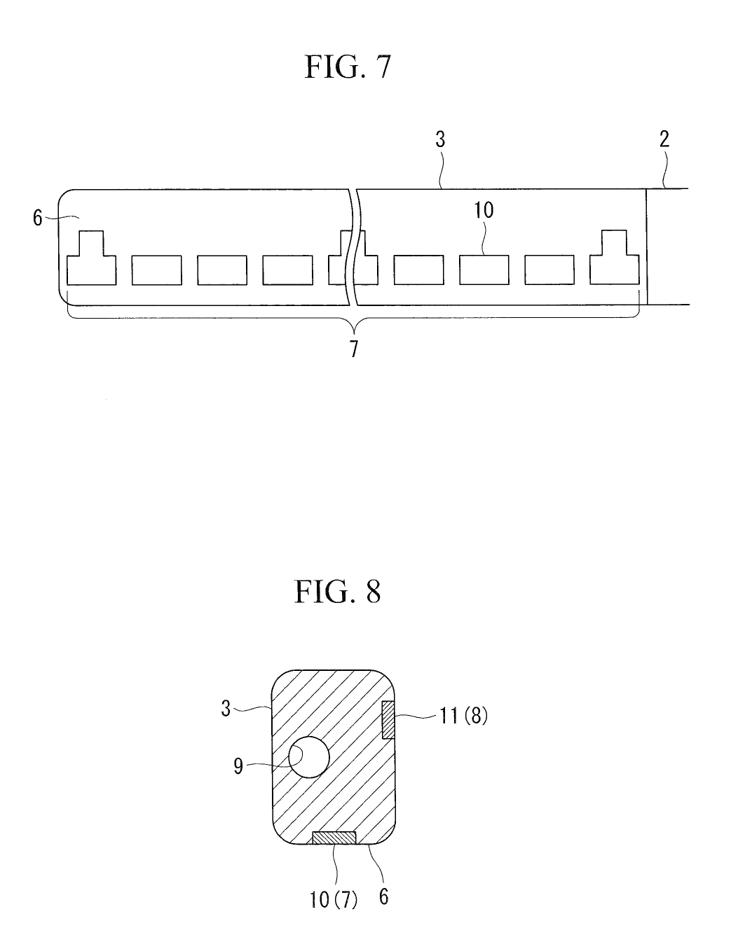

In a modification shown in FIG. 7, the marking section 8 is omitted, and at least one or more of the plurality of electrodes 10 have an asymmetrical shape in the width direction. Therefore, the orientation of the distal end section 3 can be recognized based on the projection image of the cauterizing section 7 alone in the X-ray fluoroscopic image. Accordingly, since it is not necessary to provide the marking section 8 separately from the cauterizing section 7, a simplified structure can be achieved.

Although the marking section 8 is provided on the same cauterizing surface 6 as the cauterizing section 7 in this embodiment, the disposition of the marking section 8 is not limited to this. FIGS. 8 to 11 illustrate modifications of the disposition of the marking section 8.

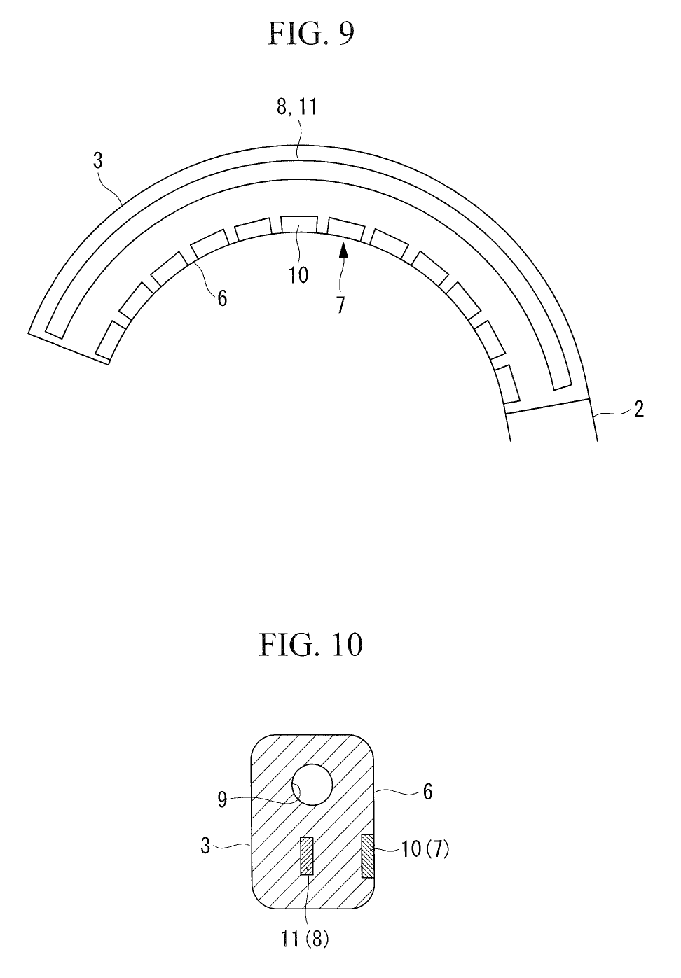

In a modification shown in FIG. 8, the marking section 8 is provided on a flat surface that is adjacent to the cauterizing surface 6 in the circumferential direction and that is substantially orthogonal to the cauterizing surface 6. Such a distal end section 3 is preferably used when linearly cauterizing an area near the pulmonary vein in treatment of auricular fibrillation. Specifically, as shown in FIG. 9, the distal end section 3 is bent so that the cauterizing surface 6 is positioned at the inner side of the bent shape, and the left atrium is cauterized in a state where the cauterizing surface 6 is wrapped around the area near the pulmonary vein of the left atrium so that the cauterizing surface 6 comes into contact with the left atrium.

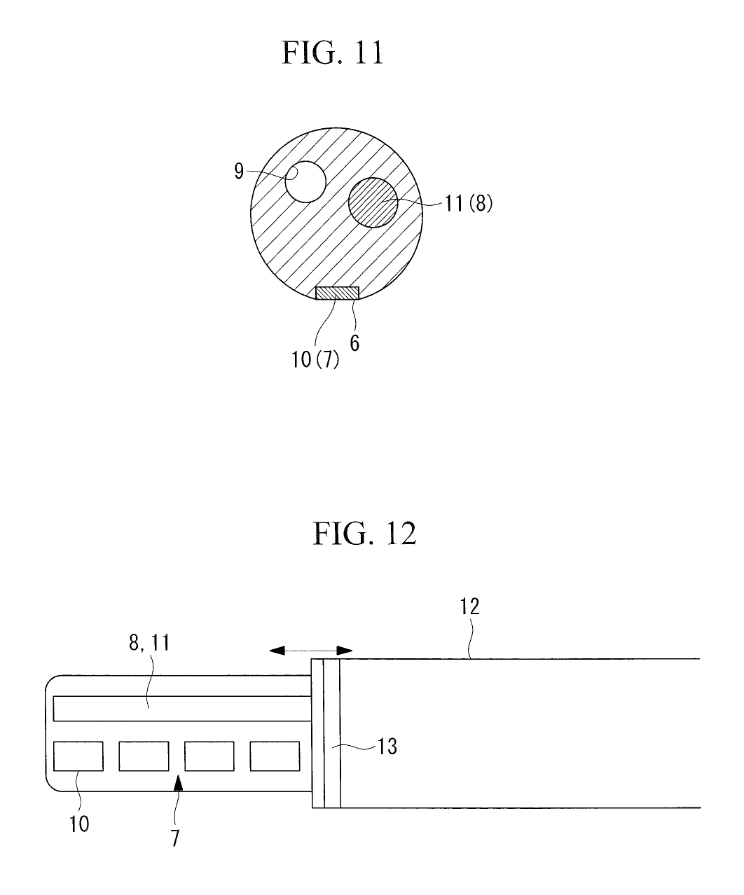

In a modification shown in FIGS. 10 and 11, the marking section 8 is provided inside the distal end section 3 without being externally exposed from the distal end section 3. As shown in FIG. 11, the distal end section 3 and the marker 11 may have a circular shape in cross section.

In this embodiment, a tubular insulation member 12 that accommodates the insertion section 2 may be further provided, as shown in FIG. 12.

The insulation member 12 has an inner diameter slightly larger than the outer diameters of the insertion section 2 and the distal end section 3 and accommodates the insertion section 2 and the distal end section 3 in a movable manner in the longitudinal direction. The insulation member 12 has electrical insulation properties and blocks high-frequency current supplied to the electrodes 10 positioned inside the insulation member 12. A marker 13 composed of a radiopaque material is provided near the distal end of the insulation member 12, so that the position of the distal end of the insulation member 12 inside the body can be recognized in the X-ray fluoroscopic image.

The insulation member 12 is disposed relative to the insertion section 2 and the distal end section 3 at a position where one or more electrodes located toward the base end among the plurality of electrodes 10 are hidden relative to surrounding tissue so that the length of a region to be cauterized can be changed. Moreover, a desired region to be treated can be selectively cauterized while using the insulation member 12 to protect tissue adjoining the region to be treated from the high-frequency current.

Although the cauterizing section 7 is provided with the electrodes 10 for high-frequency ablation in this embodiment, the cauterizing section 7 may alternatively be provided with a heating element for thermal ablation or a cooling element for cryoablation. If a heating element or a cooling element is used, the heating element or the cooling element may be provided inside the distal end section 3, as shown in FIGS. 10 and 11, since the heating element or the cooling element only needs to transmit the high-temperature heat or the low-temperature heat therefrom to tissue that is in contact with the cauterizing surface 6.

As a result, the following aspect is read by the above described embodiment of the present invention.

A first aspect of the present invention provides an ablation device including a narrow insertion section insertable into a body, a distal end section provided at a distal end of the insertion section and having a narrow cauterizing surface that is formed in a longitudinal direction in one area in a circumferential direction and that releases energy to biological tissue, and a radiopaque marking section provided at the distal end section and substantially parallel to the cauterizing surface. The marking section has a three-dimensional shape such that projection shapes obtained when projected from different sides in a radial direction of the distal end section are different from each other.

According to the aspect of the present invention, tissue can be linearly cauterized along the narrow cauterizing surface by percutaneously inserting the insertion section into the body and releasing energy from the cauterizing surface.

In this case, a surgeon operates the insertion section while using an X-ray fluoroscope to observe the radiopaque marking section provided at the distal end section and substantially parallel to the cauterizing surface. The shape of a projection image of the marking section in an X-ray fluoroscopic image varies depending on the orientation of the distal end section (i.e., the rotational angle around the longitudinal axis of the distal end section) relative to the direction of observation using the X-ray fluoroscope. Therefore, the surgeon can externally recognize the orientation of the cauterizing surface inside the body based on the shape of the projection image of the marking section in the X-ray fluoroscopic image and properly adjust the orientation of the distal end section so as to bring the cauterizing surface into contact with the tissue, whereby the tissue can be reliably cauterized.

In the above aspect, the marking section may be provided at the cauterizing surface or may be provided at a side surface of the distal end section that is substantially orthogonal to the cauterizing surface.

Accordingly, the marking section can be suitably disposed in accordance with the intended use.

In the above aspect, the marking section may include a plurality of markers arranged in the longitudinal direction in a row at an intervals.

Accordingly, the markers can be used as a scale indicating positions in the longitudinal direction of the distal end section so that it can be quantitatively recognized whether the cauterizing surface is in contact with the tissue at any of the positions of the distal end section in the longitudinal direction or whether the cauterizing surface is not in contact with the tissue.

In the above aspect, the ablation device may further include an insulation member having electrical insulation properties and accommodating the distal end section and the insertion section in a movable manner in the longitudinal direction.

Accordingly, by accommodating a base-end section of the cauterizing surface within the insulation member, the length of a region to be cauterized can be changed. Moreover, a desired region can be selectively cauterized while protecting an adjoining region from the high-frequency current.

### REFERENCE SIGNS LIST

1 ablation device
2 insertion section
3 distal end section
4 handle
5 power supply unit
6 cauterizing surface
7 cauterizing section (marking section)
8 marking section
9 guide wire hole
10 electrode
11 marker
12 insulation member

1. An ablation device comprising:
a narrow insertion section insertable into a body;
a distal end section provided at a distal end of the insertion section and having a narrow cauterizing surface that is formed in a longitudinal direction in one area in a circumferential direction and that releases energy to biological tissue; and
a radiopaque marking section provided at the distal end section and substantially parallel to the cauterizing surface,
wherein the marking section has a three-dimensional shape such that projection shapes obtained when projected from different sides in a radial direction of the distal end section are different from each other.

2. The ablation device according to claim 1,
wherein the marking section is provided at the cauterizing surface.

3. The ablation device according to claim 1,
wherein the marking section is provided at a side surface of the distal end section that is substantially orthogonal to the cauterizing surface.

4. The ablation device according to claim 1,
wherein the marking section includes a plurality of markers arranged in the longitudinal direction in a row at a intervals.

5. The ablation device according to claim 1, further comprising:
an insulation member having electrical insulation properties and accommodating the distal end section and the insertion section in a movable manner in the longitudinal direction.
